(19) United States
(12) Patent Application Publication
(10) Pub. No.: US 2005/0250706 A1
(43) Pub. Date: Nov. 10, 2005

(54) Processes for the preparation of alpha polymorph of perindopril erbumine

(75) Inventors: Narendra Shriram Joshi, Navi Mumbai (IN); Shekhar Bhaskar Bhirud, Navi Mumbai (IN); Kodali Eswara Rao, Navi Mumbai (IN)

Correspondence Address: DILWORTH & BARRESE, LLP, 333 EARLE OVINGTON BLVD., UNIONDALE, NY, 11553, US

(73) Assignee: Glenmark Pharmaceuticals Limited, Mumbai (IN)

(21) Appl. No.: 11/122,731

(22) Filed: May 5, 2005

Related U.S. Application Data
(60) Provisional application No. 60/572,402, filed on May 19, 2004.

(30) Foreign Application Priority Data
May 7, 2004 (IN) 531/MUM/2004

(51) Int. Cl. A61K 31/405; A61K 38/04; C07D 209/42

(52) U.S. Cl. 514/19; 514/419; 548/492

(57) ABSTRACT

Processes for the preparation of an alpha polymorph of perindopril erbumine is provided comprising (a) forming a solution comprising perindopril erbumine in one or more ketones; (b) heating the solution to reflux; and (c) cooling the solution to a temperature sufficient to form the alpha polymorph of perindopril erbumine. The alpha polymorphs of perindopril erbumine obtained herein have a high purity level.

### PRIORITY

This application claims the benefit under 35 U.S.C. §119 to Provisional Application No. 60/572,402, filed May 19, 2004 and entitled “PROCESS FOR THE PREPARATION OF ALPHA POLYMORPHS OF PERINDOPRIL ERBUMINE”, and from Indian Provisional Application Number 531/MUM/2004, filed May 7, 2004, the contents of which are incorporated by reference herein.

### BACKGROUND OF THE INVENTION

1. Technical Field

The present invention generally relates to improved processes for the preparation of the alpha crystalline form of perindopril erbumine.

2. Description of the Related Art

The present invention relates to a process for the preparation of perindopril erbumine (also known as (2S,3 μS,7 μS)-1-[(S)-N-[(S)-1-carboxy-butyl]alanyl]hexahydro-2-indolinecarboxylic acid, 1-ethyl ester, compound with tert-butylamine (1:1)) of Formula I: 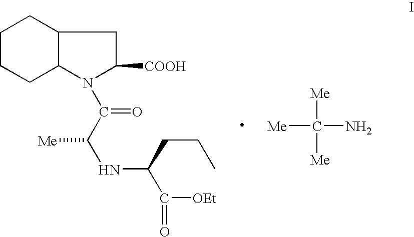
The tert-butylamine salt of perindopril, also known as perindopril erbumine, is the form commercially sold under the trade name Aceon®. Perindopril is the free acid form of perindopril erbumine and is an ethyl ester of a non-sulfhydryl angiotensin-converting enzyme (ACE) inhibitor. Perindopril is a pro-drug and is metabolized in vivo by hydrolysis of the ester group to form perindoprilat, the biologically active metabolite. Perindopril is ordinarily used to treat hypertension.

It is believed that perindoprilat lowers blood pressure primarily through inhibition of ACE activity. ACE is a peptidyl dipeptidase that catalyzes conversion of the inactive decapeptide, angiotensin I, to the vasoconstrictor, angiotensin II. Angiotensin II is a potent peripheral vasoconstrictor, which stimulates aldosterone secretion by the adrenal cortex, and provides negative feedback on renin secretion. Inhibition of ACE results in decreased plasma angiotensin II, leading to decreased vasoconstriction, increased plasma renin activity and decreased aldosterone secretion. The latter results in diuresis and natriuresis and may be associated with a small increase of serum potassium.

WO 01/87835 and U.S. Patent Application Publication No. 2005/0059609 disclose the alpha form of perindopril erbumine and a process for its preparation, the contents of which are incorporated by reference herein. The process includes using ethyl acetate as a solvent for crystallization. Problems associated with this process includes (1) low yield of the alpha polymorph of perindopril erbumine due to impurities; (2) extra crystallization using ethyl acetate to get acceptable yields of the alpha form of perindopril erbumine; and (3) a relatively large number of impurities are formed during the erbumine salt formation in ethyl acetate at reflux temperature.

Accordingly, there remains a need for improved processes for preparing the alpha crystalline form of perindopril erbumine to provide better yields and fewer impurities than the prior art processes in a convenient and more cost efficient manner on a commercial scale.

### SUMMARY OF THE INVENTION

In accordance with one embodiment of the present invention, a process for the preparation of the alpha crystalline form of perindopril erbumine of formula I: 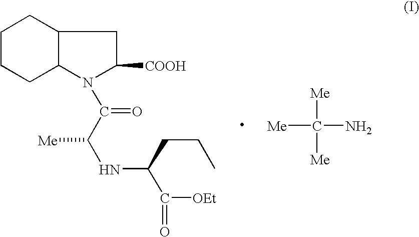
is provided comprising:
(a) forming a solution comprising perindopril erbumine of formula I in one or more ketones;
(b) heating the solution to reflux; and,
(c) cooling the solution to a temperature sufficient to form the alpha crystalline form of perindopril erbumine.

In accordance with a second embodiment of the present invention, a process for the preparation of the alpha crystalline form of perindopril erbumine of formula I is provided comprising:
(a) forming a solution comprising perindopril erbumine of formula I in one or more alcohols, one or more nitriles and one or more ketones;
(b) heating the solution to reflux; and
(c) cooling the solution to a temperature sufficient to form the alpha polymorph of perindopril erbumine.

In accordance with another embodiment of the present invention, an alpha crystalline form of perindopril erbumine of formula I having a purity of greater than about 95% is provided.

The advantages of the processes of the present invention include at least:
1. improved yields of the alpha crystalline form of perindopril; and
2. production of fewer impurities in the resulting alpha crystalline form of perindopril.

### DETAILED DESCRIPTION OF THE PREFERRED EMBODIMENTS

The present invention is directed towards improved processes for the preparation of the alpha crystalline form of perindopril erbumine of formula I: 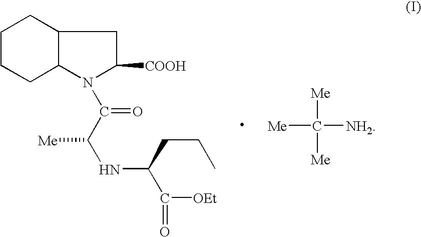
In one embodiment, the alpha crystalline form of perindopril erbumine of formula I of this invention can be obtained by (a) forming a solution comprising perindopril erbumine of formula I in one or more ketones; (b) heating the solution to reflux; and (c) cooling the solution to a temperature sufficient to form the alpha polymorph of perindopril erbumine.

Processes for the preparation of perindopril erbumine are known. See, e.g., U.S. Pat. No. 4,914,214, the contents of which are herein incorporated by reference. Generally, perindopril erbumine can be obtained by first hydrogenating indoline-2-carboxylic acid (1) in methanol over a rhodium-aluminum oxide (Rh/Al₂O₃) catalyst to form (2S,3aS,7aS)-octahydroindole-2-carboxylic acid of the formula (2) and then esterifying the acid of formula (2) with thionyl chloride and benzyl alcohol to yield (2S,3aS,7aS)-octahydroindole-2-carboxylic acid benzyl ester (3); which is one key intermediate of perindopril. Another key intermediate of perindopril is prepared by reacting L-norvaline (4) with thionyl chloride and ethanol to form an ethyl ester (5); reacting the ethyl ester (5) with sodium pyruvate (6) and subjecting the reaction product to hydrogenation to form N-1S-carboxyethylbutyl-(S)-alanine (7). Next, (2S,3aS,7aS)-octahydroindole-2-carboxylic acid benzyl ester (3) is then coupled with N-1S-carboxyethylbutyl-(S)-alanine (7) in presence of sodium diethyldithiocarbamate trihydrate (DCC) to yield perindopril benzylated ester (8). Perindopril benzylated ester (8) is hydrolyzed to form perindopril (9) which is reacted with tert-butylamine in ethyl acetate to form the perindopril erbumine salt (I). This reaction scheme is generally shown below in Scheme I:

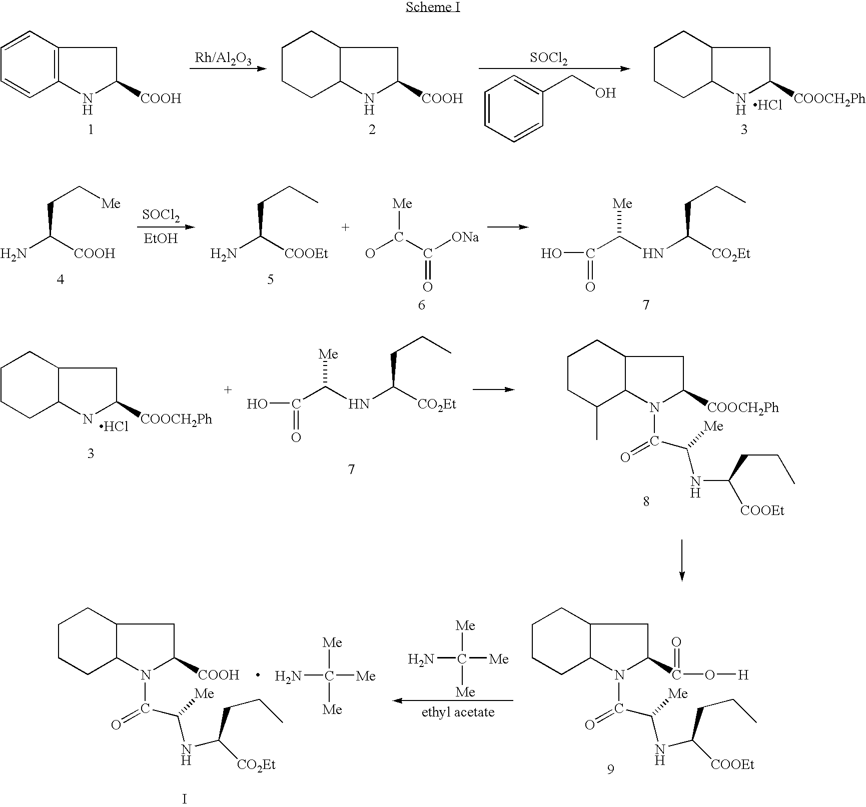

Ketones for use herein can be one or more ketones of the general formula R¹R²C(O) wherein R¹ and R² are the same or different and can be a substituted or unsubstituted C₁-C₃₀ alkyl, a substituted or unsubstituted C₃-C₃₀ cycloalkyl, a substituted or unsubstituted C₃-C₃₀ cycloalkylalkyl, a substituted or unsubstituted C₃-C₃₀ cycloalkenyl, a substituted or unsubstituted C₅-C₃₀ aryl, a substituted or unsubstituted a C₅-C₃₀ arylalkyl, a substituted or unsubstituted C₅-C₃₀ heteroaryl, a substituted or unsubstituted C₃-C₃₀ heterocyclic ring, a substituted or unsubstituted C₄-C₃₀ heterocyclylalkyl, a substituted or unsubstituted C₆-C₃₀ heteroarylalkyl; or R¹ and R² together with the carbon atom to which they are bonded are joined together to form a ring optionally containing one or more heterocyclic atoms. In another embodiment, the ketones for use herein include those containing at least three carbon atoms. Representative examples of ketones for use herein include, but are not limited to, acetone, methyl ethyl ketone, diethyl ketone, methyl propyl ketone, methyl isopropyl ketone, ethyl propyl ketone, ethyl isopropyl ketone, dipropyl ketone, diisopropyl ketone, methyl butyl ketone, methyl isobutyl ketone, methyl sec butyl ketone, methyl tert-butyl ketone, ethyl butyl ketone, ethyl isobutyl ketone, ethyl sec-butyl ketone, ethyl tert-butyl ketone, propyl butyl ketone, isopropyl butyl ketone, propyl isobutyl ketone, propyl sec-butyl ketone, propyl tert butyl ketone, isopropyl isobutyl ketone, isopropyl sec-butyl ketone, isopropyl tert-butyl ketone, dibutyl ketone, diisobutyl ketone, di-sec-butyl ketone, di-tert-butyl ketone, butyl isobutyl ketone, butyl sec-butyl ketone, butyl tert-butyl ketone, isobutyl sec-butyl ketone, isobutyl tert-butyl ketone, sec-butyl tert-butyl ketone, 5-heptanone, 5-methyl-2-hexanone (methyl isoamyl ketone), 4-methyl-2-hexanone, 3-methyl-2-hexanone, 3,4-dimethyl-2-pentanone, 3,3-dimethyl-2-pentanone, 4,4-dimethyl-2-pentanone, 3-octanone, 4-methyl-3-heptanone, 5-methyl-3-heptanone, 6-methyl-3-heptanone, 4,4-dimethyl-3-hexanone, 4,5-dimethyl-3-hexanone, 5,5-dimethyl-3-hexanone, 4-nonanone, 5-methyl-4-octanone, 6-methyl-4-octanone, 7-methyl-4-octanone, 5,5-dimethyl-4-neptanone, 5,6-dimethyl-4-heptanone, 6,6-dimethyl-4-heptanone, 2-undecanone, cyclopropanone, cyclobutanone, cyclopentanone, cyclohexanone, cycloheptanone, cyclooctanone, cyclononanone, cyclodecanone, cycloundecanone, cyclododecanone and the like and combinations thereof. A preferred ketone for use herein is acetone. Generally, the perindopril erbumine will be present in the solution at a concentration of from about 5% to about 50% weight/volume (w/v) and preferably from about 10% to about 20% w/v. The concentration of the one or more ketone present in the solution can range from about 10% to about 50% w/v and preferably from about 20% to about 50% w/v.

In another embodiment, the solution containing perindopril erbumine and one or more ketones can further include one or more alcohols, one or more nitriles and mixtures thereof. Alcohols for use herein include C₁-C₃₀ aliphatic alcohols, C₆-C₃₀ aromatic alcohols and the like and mixtures thereof. Examples of useful alcohols include, but are not limited to, methyl alcohol, ethyl alcohol, propyl alcohol, isopropyl alcohol, butyl alcohol, benzyl alcohol and the like and mixtures thereof. A preferred alcohol for use herein is isopropyl alcohol.

Nitriles for use herein include, but are not limited to, saturated or unsaturated aliphatic, alicyclic, or aromatic compounds containing a nitrile group. Included within the nitriles are compounds containing heteroatom such as those selected from Groups 13, 14, 15, 16 and 17 of the Periodic Table of Elements. Examples of nitriles for use herein include, but are not limited to, acetonitrile; propionitrile; isopropionitrile; butyronitrile; isobutyronitrile; valeronitrile; isovaleronitrile; trimethylacetonitrile; hexanenitrile; heptanenitrile; heptyl cyanide; octyl cyanide; undecanenitrile; malononitrile; succinonitrile; glutaronitrile; adiponitrile; sebaconitrile; allyl cyanide; acrylonitrile; crotononitrile; methacrylonitrile; fumaronitrile; tetracyanoethylene; cyclopentanecarbonitrile; cyclohexanecarbonitrile; dichloroacetonitrile; fluoroacetonitrile; trichloroacetonitrile; benzonitrile; benzyl cyanide; 2-methylbenzyl cyanide; 2-chlorobenzonitrile; 3-chlorobenzonitrile; 4-chlorobenzonitrile; o-tolunitrile; m-tolunitrile; p-tolunitrile and the like and mixtures thereof. A preferred nitrile for use herein is acetonitrile.

The mixture of solvents of the foregoing alcohols, nitrites and ketone may be present in the solution in a volume ratio of alcohols to nitriles to ketones of about 1:2:2. Generally, the concentration of perindopril erbumine with the foregoing solvents will range from about 10% to about 15% w/v.

In the processes of the present invention, the alpha crystalline form of perindopril erbumine is formed by dissolving perindopril erbumine in one or more ketones or a mixture of solvents comprised of the foregoing ketones, alcohols, and nitrites to form a solution. The solution is then heated to reflux, e.g., a temperature ranging from about 55° C. to about 75° C., until the solution becomes substantially clear. The solution is then cooled to a temperature sufficient to form the alpha crystalline form of perindopril erbumine. Generally, the heated solution is gradually cooled to a temperature ranging from about 25° C. to about 30° C. After cooling, the perindopril erbumine alpha polymorph is present as a crystallized white solid, which can then be filtered off by conventional techniques.

Another aspect of the present invention provides an improved process for the preparation of the alpha form of perindopril erbumine. The process involves at least (1) reacting perindopril with tert-butylamine in a halogenated hydrocarbon solvent to form perindopril erbumine; (2) forming a solution including at least perindopril erbumine of formula I in one or more of the foregoing ketones and optionally one or more of the foregoing alcohols and/or nitriles; (3) heating the solution to reflux; and (4) cooling the solution to a temperature sufficient to form the alpha polymorph of perindopril erbumine. Examples of such halogenated hydrocarbon solvents include, but are not limited to, aliphatic chlorinated solvents such as, for example, methylene chloride (also known as dichloromethane or methylene dichloride), ethylene dichloride, trichloroethylene, and chloroform; and the like and mixtures thereof with methylene chloride being the preferred halogenated hydrocarbon solvent.

The alpha crystalline form of perindopril erbumine obtained from the processes of the present invention will be in relatively high purity, e.g., a purity greater than about 95% and preferably greater than about 99%. The present invention also provides pharmaceutical compositions (formulations) containing an effective amount of the highly purified alpha crystalline form of perindopril erbumine in combination with, for example, one or more pharmaceutically acceptable carriers, excipients, diluents or adjuvants in accordance with known and established practice. For example, the highly purified alpha crystalline form of perindopril erbumine may be formulated for oral, parenteral (intravenous or subcutaneous) or nasal administration, tablets, sublingual tablets, gelatin capsules, lozenges, suppositories, creams, ointments, dermal gels, injectable preparations, drinkable suspensions etc. The useful dosage can be varied according such factors as the nature and severity of the disorder, the administration route and the age and weight of the patient. It may vary from 1 to 500 mg per day in one or more administrations.

Thus, the alpha crystalline form of perindopril erbumine of the invention can be formulated as a liquid, powder, elixir, injectable solution, etc. Formulations for oral use can be provided as tablets or hard capsules wherein the pharmacologically active ingredient(s) are mixed with an inert solid diluent such as calcium carbonate, calcium phosphate or kaolin, or as soft gelatin capsules wherein the active ingredients are mixed with water or miscible solvents such as propylene glycol (PEG); mixtures of PEG's and alcohols, e.g., ethanol, or an oleaginous medium, e.g., peanut oil; liquid paraffin or olive oil.

For topical administration in the mouth, the alpha crystalline form of perindopril erbumine of the invention can take the form of buccal or sublingual tablet, drops or lozenges formulated in conventional manner.

For topical administration to the epidermis, the alpha crystalline form of perindopril erbumine of the invention can be formulated as creams, gels, ointments or lotions or as transdermal patches. Such compositions can, for example, be formulated with an aqueous or oily base with the addition of suitable thickening, gelling, emulsifying, stabilizing, dispersing, suspending, and/or coloring agents.

The alpha crystalline form of perindopril erbumine of the invention can also be formulated as depot preparations. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example as a sparingly soluble salt.

The alpha crystalline form of perindopril erbumine of the invention can be formulated for parenteral administration by injection, conveniently intravenous, intramuscular or subcutaneous injection, for example by bolus injection or continuous intravenous infusion. Formulations for injection can be presented in unit dosage from e.g. in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient may be in powder form for constitution with a suitable vehicle, e.g. sterile pyrogen-free water, before use.

The alpha crystalline form of perindopril erbumine of the invention can also be formulated in rectal compositions such as suppositories or retention enemas. e.g. containing conventional suppository bases such as cocoa butter or other glyceride.

For intranasal administration, the alpha crystalline form of perindopril erbumine of the invention can be used, for example, as a liquid spray, as a powder or in the form of drops.

For administration by inhalation, the alpha crystalline form of perindopril erbumine of the invention can be conveniently delivered in the form of an aerosol spray presentation from pressurized packs or a nebulizer, with the use of a suitable propellant, e.g. dichlorodifluoromethane, trichlorofluoromethane, dichlorotetrafluoroethane, tetrafluoroethane, heptafluoropropane, carbon dioxide or other suitable gas. In the case of a pressurized aerosol the dosage unit can be determined by providing a valve to deliver a metered amount. Capsules and cartridges of e.g. gelatin for use in an inhaler or insulator can be formulated containing a powder mix of a compound of the invention and a suitable powder base such as lactose or starch.

Aqueous suspensions can include pharmaceutically acceptable excipients such as suspending agents, e.g., sodium carboxymethyl cellulose, methylcellulose, hydroxypropylmethylcellulose, sodium alginate, polyvinylpyrrolidone, gum tragacanth and gum acacia; dispersing or wetting agents such as naturally occurring phosphatide, e.g., lecithin, or condensation products of an alkylene oxide with fatty acids, e.g., polyoxyethylene stearate, or condensation products of ethylene oxide with long chain aliphatic alcohols, e.g, heptadecaethylene-oxycetanol, or condensation products of ethylene oxide with partial esters derived from fatty acids and a hexitol, e.g., polyoxyethylene sorbitol monoleate or condensation products of ethylene oxide with partial esters derived from fatty acids and hexitol anhydrides, e.g., polyoxyethylene sorbitan monoleate. The aqueous suspensions can also contain one or more preservatives, e.g., ethyl- or -n-propyl-p-hydroxy benzoate, one or more coloring agents, one or more flavoring agents and one or more sweetening agents, such as sucrose, saccharin or sodium or calcium cyclamate.

The following examples are provided to enable one skilled in the art to practice the invention and are merely illustrative of the invention. The examples should not be read as limiting the scope of the invention as defined in the claims.

### EXAMPLE 1

Step I: Preparation of Perindopril Erbumine

In a 4-necked round bottom flask, methylene chloride (750 ml) and N-1(S) carboxyethylbutyl-(S)-alanine (70.0 g, 0.32M) were added and cooled to about 10° C. To this N,N′-carbonyl diimidazole (96.5 g, 0.59M) was added slowly over about 45 minutes. The reaction mass was then stirred for about 2 hours at a temperature ranging from about 10° C. to about 15° C. After cooling to a temperature of about 0° C., octahydroindole-1H-2-carboxylic acid (50 g, 0.295M), was added in a single lot by maintaining temperature ranging from about 0° C. to about 5° C. and stirred for about 2 hours. The methylene chloride was then distilled off completely under vacuum below 30° C. to get an oily residue. To this oily residue, a mixture of acetic acid (36.5 g) in water (500 ml) was added at a temperature below 5° C. and stirred for 30 minutes. The reaction mass was extracted with methylene chloride (500 ml). The methylene chloride layer was then washed with saturated brine solution (100 ml) and dried over sodium sulfate (50 g). The methylene chloride layer was charged in a round bottom flask and cooled to a temperature of about 5° C. Tert-butylamine (32.5 ml) was charged to the reaction mass over 30 minuets at a temperature below 1° C. and stirred for 30 minutes. The methylene chloride is then distilled off to provide an oily residue of perindopril erbumine.

Step II: Preparation of Alpha Crystalline Form of Perindopril Erbumine

A mixture of isopropyl alcohol (150 ml), acetone (300 ml) and acetonitrile (300 ml) was charged to the oily residue obtained in step I and heated to a temperature ranging from about 65° C. to about 70° C. to obtain a clear solution. The reaction mass was then cooled very slowly to about 25° C. over about 2 hours. The reaction mass was then further cooled to about 0° C. and a white solid was filtered. The white solid was then dried under vacuum at about 40° C. (Weight: 87.6 g, %-yield: 90%, purity: >99.5% by HPLC). Specific optical rotation [α]n=−66 (C=1%, MeOH), IR (KBr) spectrum shows the following absorptions cm−1 3300, 2930, 1744, 1732 m 1644 m 1568. The 1H-NMR (CDCl3) shows the following signals at δ 4.28-4.12 (m, 1H), 4.18-4.09 (q, 2H), 3.76 (m, 2H) 3.53 (q, 1H), 3.1 (t, 1H), 2.32-2.14 (m, 2H), 2.01 (m, 1H), 1.75-1.62 (m, 4H), 1.32 (m, 2H), 1.30 (S, 9H), 1.28 (t, 3H), 0.88 (t, 3H). CI Mass shows m/z at 368 (base peak). The x-ray diffraction data for this example was performed using a Shimadzu XRD-6000. The x-ray diffraction data is set forth below in Table 1.

TABLE 1

| Angle 2θ(°) | d(A) | I/II |
|---|---|---|
| 8.34 | 10.59 | 5 |
| 8.92 | 9.90 | 100 |
| 10.82 | 8.17 | 13 |
| 15.57 | 6.52 | 5 |
| 14.46 | 6.12 | 33 |
| 15.18 | 5.83 | 62 |
| 16.36 | 5.41 | 77 |
| 17.18 | 5.16 | 28 |
| 17.91 | 4.95 | 7 |
| 19.77 | 4.49 | 18 |
| 20.40 | 4.35 | 30 |
| 21.30 | 4.16 | 14 |
| 21.98 | 4.04 | 37 |
| 22.42 | 3.96 | 29 |
| 23.98 | 3.71 | 25 |
| 24.56 | 3.62 | 7 |
| 25.65 | 3.47 | 9 |
| 27.56 | 3.23 | 11 |
| 28.81 | 3.09 | 6 |

### EXAMPLE 2

### Preparation of Pharmaceutical Tablets Containing the Alpha Crystalline Form of Perindopril Erbumine

The ingredients for use in this example are set forth below in Table 2. The product was made by standard tabletting process as disclosed in, for example, Ansel et al., Pharmaceutical Dosage Forms and Drug Delivery Systems 7th Edition (1999), and incorporating the alpha crystalline form of perindopril erbumine of Example 1.

TABLE 2

| | Mg/tablet | | |
|---|---|---|---|
| Ingredients | For 2 mg | For 4 mg | For 8 mg |
| Alpha Crystalline Form | 2.0 | 4.0 | 8.0 |
| of Perindopril | | | |
| Erbumine | | | |
| Hydrophobic colloidal | 0.135 | 0.27 | 0.54 |
| silicon dioxide | | | |
| (Aerosil R972) | | | |
| MCC (Avicel PH 112) | 11.25 | 22.5 | 45.0 |
| Lactose monohydrate | 31.39 | 62.78 | 125.56 |
| (Tablettose 100) | | | |
| Magnesium Stearate | 0.225 | 0.45 | 0.9 |
| Average Weight/Tablet | 45.0 mg | 90.0 mg | 180.0 mg |

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

1. A process for the preparation of the alpha crystalline form of perindopril erbumine of formula I: 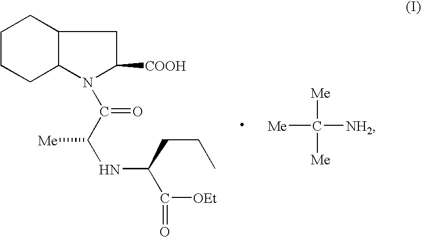
the process comprising:
(a) forming a solution comprising perindopril erbumine of formula I in one or more ketones;
(b) heating the solution to reflux; and
(c) cooling the solution to a temperature sufficient to form the alpha crystalline form of perindopril erbumine.

2. The process of claim 1, wherein the one or more ketones are of the general formula R¹R²C(O) wherein R¹ and R² are the same or different and can be a substituted or unsubstituted C₁-C₃₀ alkyl, a substituted or unsubstituted C₃-C₃₀ cycloalkyl, a substituted or unsubstituted C₃-C₃₀ cycloalkylalkyl, a substituted or unsubstituted C₃-C₃₀ cycloalkenyl, a substituted or unsubstituted C₅-C₃₀ aryl, a substituted or unsubstituted a C₅-C₃₀ arylalkyl, a substituted or unsubstituted C₅-C₃₀ heteroaryl, a substituted or unsubstituted C₃-C₃₀ heterocyclic ring, a substituted or unsubstituted C₄-C₃₀ heterocyclylalkyl, a substituted or unsubstituted C₆-C₃₀ heteroarylalkyl; or R¹ and R² together with the carbon atom to which they are bonded are joined together to form a ring optionally containing one or more heterocyclic atoms.

3. The process of claim 1, wherein the ketone is acetone.

4. The process of claim 1, wherein the concentration of the perindopril erbumine of formula I in the solution is about 5% to about 50% weight/volume (w/v).

5. The process of claim 1, wherein the concentration of the one or more ketone in the solution is about 10% to about 50% w/v.

6. The process of claim 1, wherein the step of forming the solution further comprises adding one or more alcohols and one or more nitrites.

7. The process of claim 6, wherein the one or more alcohols are selected from the group consisting of C₁-C₃₀ aliphatic alcohols, C₆-C₃₀ aromatic alcohols and mixtures thereof.

8. The process of claim 6, wherein the one or more nitriles are saturated or unsaturated aliphatic, alicyclic, or aromatic compounds containing a nitrile group and optionally containing one or more heteroatoms.

9. The process of claim 6, wherein the one or more alcohols are selected from the group consisting of methyl alcohol, ethyl alcohol, propyl alcohol, isopropyl alcohol, butyl alcohol, benzyl alcohol and mixtures thereof and the one or more nitriles are selected from the group consisting of acetonitrile; propionitrile; isopropionitrile; butyronitrile; isobutyronitrile; valeronitrile; isovaleronitrile; trimethylacetonitrile; hexanenitrile; heptanenitrile; heptyl cyanide; octyl cyanide; undecanenitrile; malononitrile; succinonitrile; glutaronitrile; adiponitrile; sebaconitrile; allyl cyanide; acrylonitrile; crotononitrile; methacrylonitrile; fumaronitrile; tetracyanoethylene; cyclopentanecarbonitrile; cyclohexanecarbonitrile; dichloroacetonitrile; fluoroacetonitrile; trichloroacetonitrile; benzonitrile; benzyl cyanide; 2-methylbenzyl cyanide; 2-chlorobenzonitrile; 3-chlorobenzonitrile; 4-chlorobenzonitrile; o-tolunitrile; m-tolunitrile; p-tolunitrile and mixtures thereof.

10. The process of claim 6, wherein the ketone is acetone, the alcohol is isopropyl alcohol and the nitrile is acetonitrile.

11. The process of claim 6, wherein the alcohol, nitrile and ketone are present in the solution in a volume ratio of about 1:2:2.

12. The process of claim 11, wherein the alcohol is isopropyl alcohol, the nitrile is acetonitrile and the ketone is acetone.

13. The process of claim 1 wherein the perindopril erbumine is obtained from the reaction of perindopril with tert-butylamine in a halogenated hydrocarbon solvent.

14. The process of claim 13, wherein the halogenated hydrocarbon solvent is methylene chloride.

15. The process of claim 1, wherein the alpha polymorph of perindopril erbumine has a purity greater than about 95%.

16. The process of claim 1, wherein the alpha polymorph of perindopril erbumine has a purity greater than about 99%.

17. The process of claim 6, wherein the alpha crystalline form of perindopril erbumine has a purity greater than about 95%.

18. An alpha crystalline form of perindopril erbumine having a purity of greater than about 95%.

19. An alpha crystalline form of perindopril erbumine having a purity of greater than about 99%.

20. An alpha crystalline form of perindopril erbumine produced by the process of claim 1.

21. An alpha crystalline form of perindopril erbumine produced by the process of claim 6.

22. A process for the preparation of perindopril erbumine comprising reacting perindopril with tert-butylamine in a halogenated hydrocarbon solvent.

23. The process of claim 22, wherein the halogenated hydrocarbon is an aliphatic chlorinated solvent.

24. The process of claim 23, wherein the aliphatic chlorinated solvent is selected from the group consisting of methylene chloride, ethylene dichloride, trichloroethylene, chloroform and mixtures thereof.

25. A process for the preparation of the alpha crystalline form of perindopril erbumine of formula I: 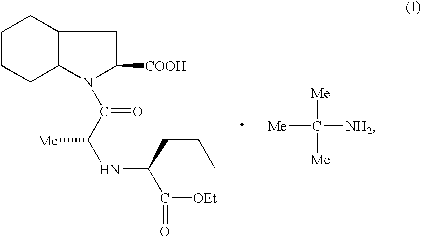
the process comprising:
(a) reacting perindopril with tert-butylamine in a halogenated hydrocarbon to form perindopril erbumine of formula I;
(b) forming a solution comprising the perindopril erbumine of formula I in one or more ketones, one or more alcohols and one or more nitrites;
(c) heating the solution to reflux; and
(d) cooling the solution to a temperature sufficient to form the alpha crystalline form of perindopril erbumine.

26. A pharmaceutical composition comprising a therapeutically effective amount of an active pharmaceutical ingredient comprising the alpha crystalline form of perindopril erbumine obtained from the process of claim 1.

27. A pharmaceutical composition comprising a therapeutically effective amount of an active pharmaceutical ingredient comprising the alpha crystalline form of perindopril erbumine obtained from the process of claim 6.

28. A pharmaceutical composition comprising a therapeutically effective amount of an active pharmaceutical ingredient comprising the alpha crystalline form of perindopril erbumine of claim 18.

29. The pharmaceutical composition of claim 28, further comprising a pharmaceutically acceptable carrier.

30. The pharmaceutical composition of claim 28, further comprising one or more pharmaceutically acceptable excipients.

31. A pharmaceutical composition comprising a therapeutically effective amount of an active pharmaceutical ingredient comprising the alpha crystalline form of perindopril erbumine of claim 19.

32. The pharmaceutical composition of claim 31, further comprising a pharmaceutically acceptable carrier.

33. The pharmaceutical composition of claim 31, further comprising one or more pharmaceutically acceptable excipients.
